(19) United States
(12) Patent Application Publication
(10) Pub. No.: US 2010/0049101 A1
(43) Pub. Date: Feb. 25, 2010

(54) Apparatus and Methods for Delivering Acoustic Energy to Body Tissue

(75) Inventor: Gopal K. Chopra, San Francisco, CA (US)

Correspondence Address: VISTA IP LAW GROUP LLP, 12930 Saratoga Avenue, Suite D-2, Saratoga, CA, 95070, US

(73) Assignee: BOSTON SCIENTIFIC SCIMED INC., Maple Grove, MN (US)

(21) Appl. No.: 12/605,981

(22) Filed: Oct. 26, 2009

Related U.S. Application Data
(63) Continuation of application No. 10/728,022, filed on Dec. 3, 2003.

(51) Int. Cl. A61N 7/00

(52) U.S. Cl. 601/2

(57) ABSTRACT

Apparatus for delivering acoustic energy to a target site includes a structure and a transducer secured to the structure, the transducer having a surface configured to be placed on a tissue. The structure comprises a channel located adjacent the transducer, the channel adapted for carrying cooling fluid.

### RELATED APPLICATION DATA

The present application is a continuation of pending U.S. patent application Ser. No. 10/728,022, filed Dec. 3, 2003, the priority of which is claimed under 35 U.S.C. §120, and the contents of which is incorporated herein by reference in its entirety, as though set forth in full.

### FIELD OF INVENTION

The invention relates generally to apparatus and methods for delivering acoustic energy to a target tissue structure or region in a body.

### BACKGROUND

Devices and systems using acoustic energy, particularly within the ultrasonic range (acoustic waves with a frequency greater than about twenty kHz, and more typically between about fifty kHz and about five MHz (0.05-5 MHz)), have been used to diagnose and treat patients. For example, ultrasonic energy may be employed to obtain images of a part of a patient during a diagnostic or therapeutic procedure. In addition, ultrasound systems have been used for treating tissue, e.g., by directing acoustic energy towards a target tissue region within a patient, such as a cancerous or benign tumor, to heat or ablate the tissue region. For example, one or more piezoelectric transducers may be disposed adjacent a patient's body and used to deliver high intensity acoustic waves, such as ultrasonic waves, at an internal tissue region of a patient to treat the tissue region. An exemplary focused ultrasound system is disclosed in U.S. Pat. No. 4,865,042 issued to Umemura et al. The acoustic energy emitted from such a system may be focused at a desired focal zone to deliver thermal energy to the target tissue region.

Ultrasonic devices have also been used to identify occluded cranial vessels, and to treat vessel occlusion and hypoperfusion in the brain. When identifying cranial vessels using transcranial Doppler ultrasound (TCD), ultrasonic energy is transmitted from a transducer to brain tissue beneath a patient's skin, and sonic waves reflected from the patient's internal tissue are analyzed using the Doppler principle to determine a location of a cranial vessel.

When treating an occluded cranial vessel, an ultrasonic transducer is placed against a patient's skin, and ultrasonic energy is directed from the transducer to the occluded cranial vessel to dilate and recannalize the occluded vessel, thereby improving oxygen delivery to brain tissue. However, unlike a diagnostic session for vessel identification, which generally takes about 10 to 15 minutes to complete, an ultrasonic treatment session for treating vessel occlusion may take from 1 to 1.5 hours. Due to the prolonged application of ultrasonic energy, a patient may experience a burning sensation at the skin where the transducer is placed. The discomfort associated with the burning sensation can become so severe that the treatment session may be terminated and reinitiated after allowing the skin temperature to subside. In addition, the heat generated at the skin-transducer interface may cause tissue adjacent the skin to heat up, adversely impacting an effectiveness of the recannalization procedure.

### SUMMARY OF THE INVENTION

In one embodiment, an apparatus for delivering acoustic energy to a target site is provided, the apparatus including a transducer secured to a structure and configured to be placed on a tissue, the structure including a channel located adjacent the transducer and adapted for carrying cooling fluid.

In another embodiment, an apparatus for delivering acoustic energy to a tissue region includes a catheter having a transducer secured to the catheter distal end and means for cooling the catheter distal end.

In still another embodiment, a method for performing an ultrasound therapy includes placing a transducer on a tissue surface, delivering a blood thinning agent to a target tissue region below the tissue surface, delivering acoustic energy to the target tissue region to cause dilation of a vessel at the target tissue region, and cooling the tissue surface to reduce heat that is generated from the delivered acoustic energy.

Other aspects and features of the invention will be evident from reading the following detailed description of the illustrated embodiments, which are provided to illustrate, not limit, the invention.

### BRIEF DESCRIPTION OF THE DRAWINGS

The drawings illustrate the design and utility of embodiments of the invention, in which similar elements are referred to by common reference numerals, and in which:

FIG. 1 shows an ultrasonic device having an ultrasonic transducer that is carried by a helmet in accordance with one embodiment of the invention;

FIG. 2 shows the ultrasonic transducer of FIG. 1;

FIG. 3 shows a variation of the ultrasonic transducer of FIG. 1;

FIG. 4 shows an ultrasonic device having an ultrasonic transducer that is carried by an elongate member in accordance with another embodiment of the invention; and

FIG. 5 shows an ultrasonic device having an ultrasonic transducer that is carried by a handle in accordance with yet another embodiment of the invention.

### DETAILED DESCRIPTION OF THE ILLUSTRATED EMBODIMENTS

Various embodiments of the invention are described hereinafter with reference to the figures. It should be noted that the figures are not drawn to scale and that elements of similar structures or functions are represented by like reference numerals throughout the figures. It should also be noted that the figures are only intended to facilitate the description of specific embodiments of the invention. They are not intended as an exhaustive description of the invention or as a limitation on the scope of the invention. In addition, an illustrated embodiment needs not have all the aspects or advantages of the invention shown. An aspect or an advantage described in conjunction with a particular embodiment is not necessarily limited to that embodiment and can be practiced in other embodiments of the invention even if not so illustrated or described.

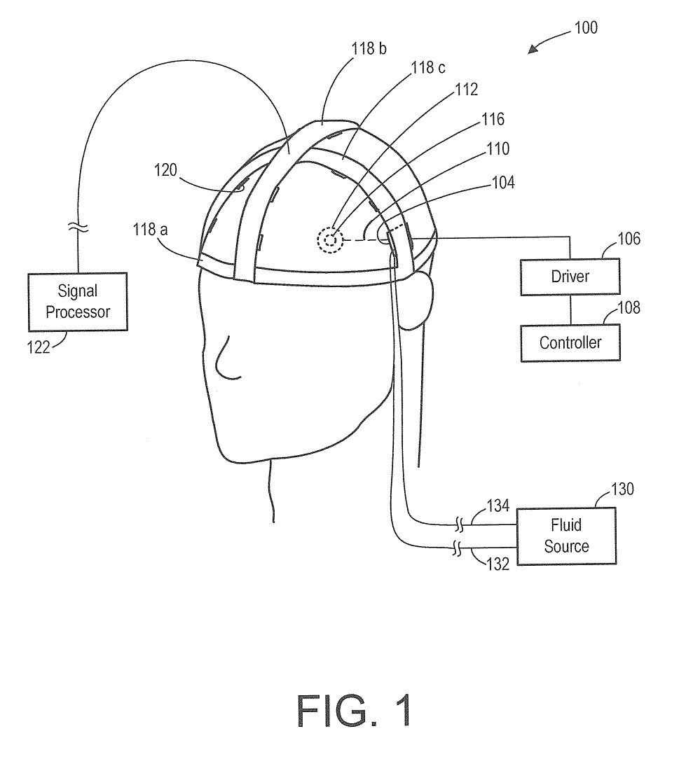

FIG. 1 shows an exemplary embodiment of a focused ultrasound device 100 including a harness 102, a transducer device 104 secured to the harness 102, a drive circuitry (driver) 106 coupled to the transducer device 104, a controller 108 coupled to the drive circuitry 106, and a fluid source 130 for delivering cooling fluid to the transducer device 104. The focused ultrasound device 100 also includes a plurality of sensors 120, such as electrodes, secured to the harness 102 for sensing neurological signals, and a signal processor 122 coupled to the sensors 120 for processing sensed neurological signals. The processor 122 can be a part of a computer, a machine, or an equipment, or alternatively, a component that is configured to be coupled to a computer.

The harness 102 is configured to be placed at least partially around a patient's head. In the illustrated embodiment, the harness 102 includes a plurality of straps 118a-c forming a helmet. The straps 118a-c can be fixedly or slidably secured to each other, and can include adjustable and securing means, such as belt holes, fastening fabric or Velcro™, and snap fit connectors, for changing a shape of the harness 102 to accommodate different head sizes. The straps 118a-c can be made from a variety of materials, such as plastics, metals, alloys, and leather. Although only three straps 118a-c are shown, in alternative embodiments, the harness 102 can have fewer or more than three straps.

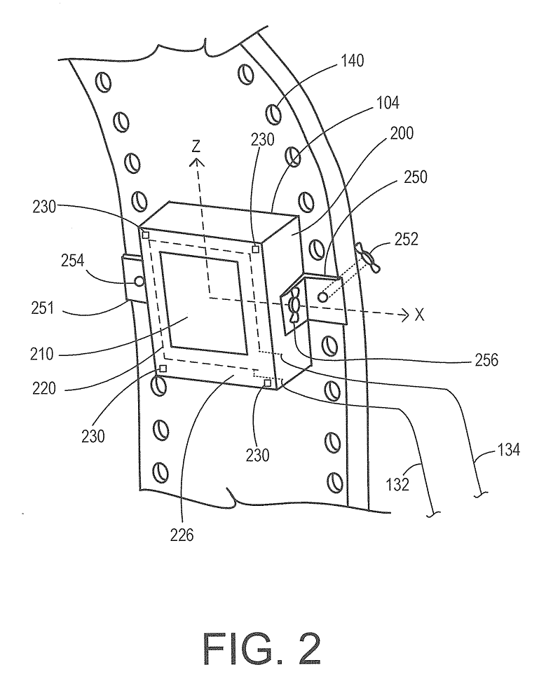

Any or all of the straps 118a-c can include a plurality of openings 140 for allowing the transducer device 104 to detachably secure to the harness 102 at different positions (FIG. 2). In the illustrated embodiment, the transducer device 104 can be detachably secured to the harness 102 by connectors 250, 251 having a first knob 252 and a second knob 254, respectively. If it is desired to change a position of the transducer device 104 relative to the harness 102, the knobs 252, 254 can be unscrewed from the connectors 250, 251 to release the transducer device 104, and are placed through different openings 140 to mate with the connectors 250, 251. In one embodiment, the knobs 252, 254 can be independently operated to adjust a rotation of the transducer device 104 about a Z-axis. The transducer 14 can also be rotatable about a X-axis, and be secured in place by a third knob 256.

It should be noted that the manner in which the transducer device 104 is secured to the harness 102 should not be limited to the example discussed previously. In alternative embodiments, other securing mechanisms, such as a snap-fit connection, a clip, a fastening fabric or Velcro™, and a frictional connection, can also be used to detachably secure the transducer device 104 to the harness 102. Also, instead of the configuration discussed previously, the transducer device 104 can be made rotatably adjustable using other attachment devices.

The transducer device 104 is configured to deliver acoustic energy (represented by beam 110) to a target tissue region 112 located within a brain. As shown in FIG. 2, the transducer device 104 includes a housing 200 and a transducer 210 secured to the housing 200. The housing 200 can be made from a variety of materials, such as polymers, plastics, metals, and alloys. The transducer 210 may be a one-piece piezoceramic part, or alternatively, be composed of a mosaic arrangement of a plurality of small piezoceramic elements. The piezoceramic parts or the piezoceramic elements may have a variety of geometric shapes, such as hexagons, triangles, squares, and the like. In one embodiment, the transducer 210 is time-delayed or phase-delayed driven. In such case, delay elements (not shown), well known in the art, may be coupled to each of the piezoceramic elements for providing delay times such that the delivered acoustic waves by the piezoceramic elements focus onto a zone. The delay elements may be implemented as a part of the ultrasound device 104, the driver 106, or the controller 108.

In the illustrated embodiment, the transducer 210 has a substantially planar surface. In alternative embodiments, the transducer 210 has a concave or bowl shape, such as a “spherical cap” shape, i.e., having a substantially constant radius of curvature such that the transducer 210 has an inside surface defining a portion of a sphere. In the illustrated embodiment, the transducer 210 has an outer perimeter that is rectangular. However, the transducer 210 can also have other shapes, such as a circular shape, an elliptical shape, or other customized shapes. The transducer 210 can also include any desired number of rings and/or sectors (not shown). In one embodiment, the transducer 210 may have an outer diameter of between about twenty and fifty millimeters (20-50 mm), a radius of curvature between about eight and sixteen centimeters (8-16 cm), and includes between ten and thirty (10-30) rings and between four and sixteen (4-16) sectors.

The transducer 210 is coupled to the driver 106 and/or controller 108 for generating and/or controlling the acoustic energy emitted by the transducer 210. For example, the driver 106 may generate one or more electronic drive signals, which may be controlled by the controller 108. The transducer 210 converts the drive signals into acoustic energy, which may be focused using conventional methods. In the illustrated embodiment, the transducer device 104 is configured to deliver focused (e.g., through a lens) or diffused acoustic energy at a frequency of between about 2 MHz to about 10 MHz sufficient to recannalize an occluded vessel. In alternative embodiments, the transducer device 104 can deliver acoustic energy at a level sufficient to heat or necrose (or otherwise treat) the target tissue region 112.

The controller 108 and/or driver 106 may be separate or integral components. It will be appreciated by one skilled in the art that the operations performed by the controller 108 and/or driver 106 may be performed by one or more controllers, processors, and/or other electronic components, including software and/or hardware components. The terms controller and control circuitry may be used herein interchangeably, and the terms driver and drive circuitry may be used herein interchangeably.

The driver 106, which may be an electrical oscillator, may generate drive signals in the ultrasound frequency spectrum, e.g., as low as twenty 20 KHz, and typically ranging from about 0.5 to 10 MHz. Preferably, the driver 106 provides drive signals to the transducer 210 at frequencies, for example, between about 7 and 10 MHz, or a more focused 2 MHz via a lens. When the drive signals are provided to the transducer 210, the transducer 210 emits acoustic energy from its exposed surface, as is known to those skilled in the art.

The controller 108 may control the amplitude, and therefore the intensity or power of the acoustic waves transmitted by the transducer 210. The controller 108 may also control a phase component of the drive signals to respective transducer elements of the transducer 210, e.g., to control a shape of a focal zone 116 generated by the transducer 210 and/or to move the focal zone 116 to a desired location. For example, the controller 108 may control the phase shift of the drive signals based upon a radial position of respective transducer elements of the transducer 210, e.g., to adjust a focal distance of the focal plane (i.e., the distance from the face of the transducer 210 to the center of the focal zone 116). In addition or alternatively, the controller 108 may control the phase shift of the drive signals based upon an angular position around the face of the transducer device 104, e.g., to adjust a shape of the focal zone 116, as is well known to those skilled in the art.

In the illustrated embodiment, the housing 200 has a channel 220 that is in fluid communication with the fluid source 130. The channel 220 is located circumferentially around the transducer 210 and is configured to circulate cooling fluid delivered from the fluid source 130. The fluid source 130 includes a cooling device (not shown), such as a heat exchanger, for cooling fluid stored therein, and a pump (also not shown) for pumping the fluid to the channel 220 via a first tube 132. The fluid travels through the channel 220, providing cooling effect to a surface 226 located adjacent to the transducer 210, and returns to the fluid source via a second tube 134.

In the illustrated embodiment, the surface 226 is approximately in a same plane with the transducer 210 such that when the transducer 210 is placed on a patient's skin, the surface 226 is also in contact with the skin. An opposite side of the surface 226 is in fluid contact with the channel 220, thereby allowing fluid in the channel 220 to carry heat away from the surface 226. In the illustrated embodiment, the surface 226 is made from a metal, or other thermally conductive materials.

In some embodiments, the transducer device 104 can further include one or more acoustic energy sensors 230 secured to the housing 200 for sensing acoustic energy or signal that has been reflected from an object, such as tissue. The sensed acoustic signal can be processed and/or analyzed to determine whether a vessel (e.g., an occluded vessel) has been located.

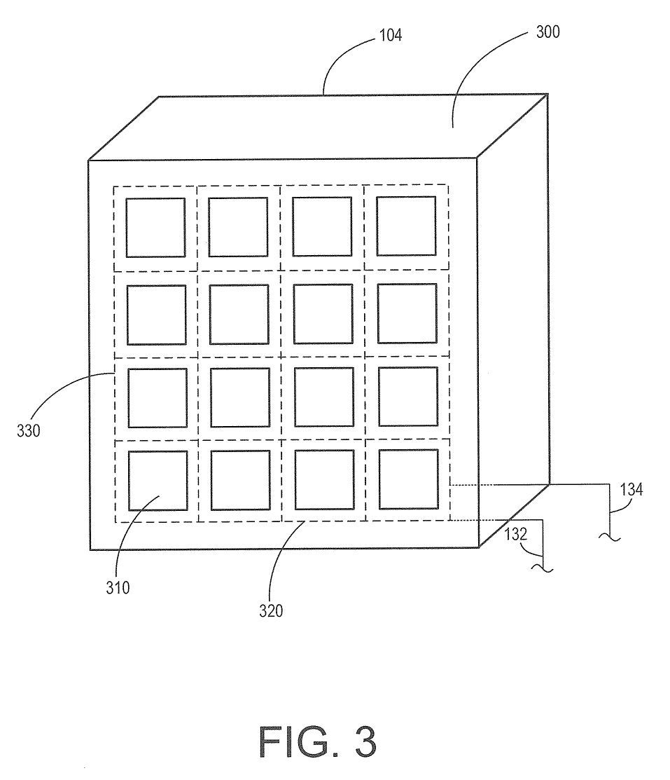

In alternative embodiments, instead of constructing the channel 220 around the transducer 210, the channel 220 can be integrated with the transducer 210. FIG. 3 shows a variation of the transducer device 104 that includes a housing 300 and a plurality of transducer elements 310 (e.g., piezoceramic elements) secured to the housing 300. The housing 300 includes a plurality of horizontal channels 320 and a plurality of vertical channels 330 forming a grid that is integrated with the transducer elements 310. The channels 320, 330 are in fluid communication with the fluid source 130. In alternative embodiments, the housing 300 can include only the horizontal channels 320 or only the vertical channels 330. Although the housing 300 has a channel 320 or 330 between each vertical and horizontal spacing of the transducer elements 310, in alternative embodiments, the housing 300 can have channels that are spaced at different rows or columns.

Furthermore, in alternative embodiments, instead of having a rectangular grid pattern, the housing 300 can have channels that form other patterns, depending on a configuration of the transducer elements 310.

When using the focused ultrasound device 100 to recannalize an occluded vessel within a brain, the transducer device 104 is first secured to the harness 102, and the harness 102 is secured to a patient's head. If necessary, the orientation of the transducer device 104 about the Z-axis and/or the X-axis can be adjusted until the transducer device 104 is aimed at a desired direction (i.e., towards a target tissue region). A coupling gel can be applied to the patient's skin to provide acoustic coupling between the patient's skin and the surface of the transducer 210. Alternatively, instead of placing the transducer 210 on the patient's skin, a burr can be created on the patient's skull, and the transducer 210 can be placed on a dura.

If the transducer device 104 includes the acoustic signal sensor, the driver 106 can generate drive signals to cause the transducer 210 to deliver acoustic energy in the diagnostic range. The acoustic signal sensor is then used to sense reflected acoustic energy, and the sensed signal is then transmitted to a processor, which determines whether the transducer 210 is aimed towards a desired target, such as a vessel. Alternatively, if the transducer device 104 does not include the sensor, then a target vessel can be located by using a separate ultrasonic device for such purpose, or by imaging techniques, such as CT imaging.

Next, a blood thinning agent is preferably (but not necessarily) administered to the patient either orally or by injection, and is allowed to react within the patient's body. Alternatively, a blood thinning agent can be administered to the patient some time before the treatment session to allow the blood thinning agent to react within the patient's body. Driver 106 then drives the transducer 210 to deliver treatment acoustic energy towards the target vessel. The acoustic energy causes the vessel to be dilated, which disrupts—and thereby facilitates, lysis of the clot. The inventor hereto believes this may be due to one or both of mechanical disruption and endogenous release of a vaso-dilatory chemical, such as nitric oxide.

While the transducer 210 applies treatment energy to the target tissue region 112, the fluid source 130 pumps cooling fluid to the channel 220 via the first tube 132. The cooling fluid travels through the channel 220 to cool the surface 226 of the transducer device 104, thereby preventing or at least reducing some of the heat that may be generated at the interface between the patient's skin and the surface of the transducer 210. The cooling fluid carries heat away from the surface 226 and travels back to the fluid source 130 via the second tube 134. Such arrangement is beneficial in that it prevents or reduces the level of discomfort typically associated with a recannalization procedure due to heat generated at the interface between a patient's skin and the transducer's surface. The cooling of the transducer device 104 is also beneficial in that it also prevents or reduces a heating of neurons due to the application of recannalization energy. In one embodiment, the fluid source 130 and the channel 220 are configured to cool the patient's skin to a temperature that is about 3 to 5° C. below a normal body temperature. In another embodiment, the fluid source 130 and the channel 220 are configured to maintain the patient's skin at normal body temperature.

While the transducer 210 applies acoustic treatment energy to the target tissue region 112, the sensors 120 can be used to sense neurological signals. The sensed neurological signals are processed by the signal processor 122, and the processed signals can be displayed in graphical form to represent neuron functions in substantially real time. Such feature is beneficial in that it allows a physician to monitor the patient's brain activity (e.g., EEG activity) to ensure that ultrasonic treatment energy is being properly applied.

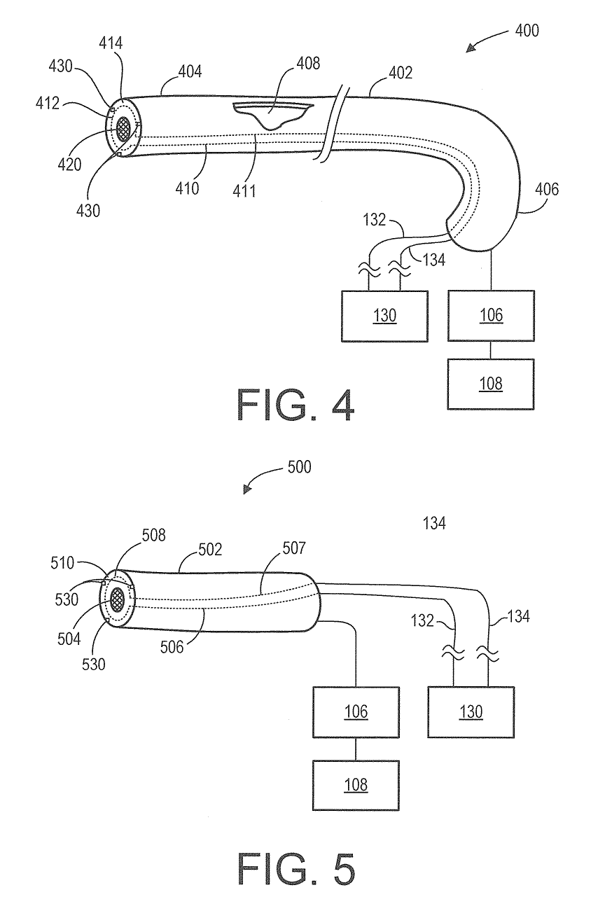

FIG. 4 shows a focused ultrasound device 400 in accordance with another embodiment of the invention. The focused ultrasound device 400 includes an elongate member 402, such as a shaft, a catheter, or a probe, having a distal end 404, a proximal end 406, and a lumen 408 extending therebetween. The elongate member 402 also includes a transducer 420 secured to the distal end 404, a first fluid delivery lumen 410, and a second fluid delivery lumen 411. The fluid delivery lumens 410, 411 extend from the proximal end 406 to the distal end 404, and fluidly couple the fluid source 130 to a channel 412 at the distal end 404. The first fluid delivery lumen 410 is configured to deliver fluid from the fluid source 130 to the distal end 404, and the second fluid delivery lumen 411 is configured to return the delivered fluid back to the fluid source 130. In the illustrated embodiment, the fluid delivery lumens 410, 411 are located within a wall of the elongate member 402. Alternatively, the fluid delivery lumens 410, 411 can be implemented using tubes that are placed within the lumen 408 of the elongate member 402. In addition, the elongate member 402 may include one or more leads (not shown), e.g., wires or conductive paths, therein coupled to the transducer 420 and extending to the proximal end 406. The proximal end 406 may include connectors (not shown) for connecting cables and the like to the elongate member 402, e.g., to couple the transducer 420 to the driver 106 and/or the controller 108. The elongate member 402 can be substantially rigid, semi-rigid, or substantially flexible, preferably having sufficient column strength such that it can be advanced into a body passage without buckling or kinking. In some embodiments, the focused ultrasound device 400 can further include a radiopaque marker secured to the distal end 404 of the elongate member 402 for allowing a visualization of the distal end 404 under imaging.

The elongate member 402 has a cross sectional dimension that allows the distal end 404 to be inserted into a body passage. The body passage may be a natural passage, such as a rectal orifice, mouth, esophagus, a nasal orifice, vagina, or a vessel. Alternatively, the body passage may be a surgically-created passage. As such, the cross sectional dimension of the elongate member may vary depending upon the particular application or surgical procedure.

In the illustrated embodiment, the elongate member 402 includes a surface 414 located at a distal tip of the elongate member 402. The surface 414 is located adjacent to the transducer 420 such that when the transducer 420 is place on a patient's skin, the surface 414 is also in contact with the skin. An opposite side of the surface 414 is in fluid contact with the channel 412, thereby allowing fluid in the channel 412 to carry heat away from the surface 414. In the illustrated embodiment, the surface 414 is made from a metal, or other thermally conductive materials.

In some embodiments, the focused ultrasound device 400 can further include one or more acoustic energy sensors 430 secured to the distal end 404 for sensing acoustic energy that has been reflected from an object, such as tissue. The sensed acoustic energy can be processed and/or analyzed to determine whether a vessel (e.g., an occluded vessel) has been identified, as similarly discussed previously.

When using the focused ultrasound device 400 to recannalize an occluded vessel, the distal end 404 is inserted into a patient, and the distal end 404 is distally advanced until it reaches a target site. Steering mechanisms known in the art can be provided to steer the distal end 404. If the device 400 includes the acoustic energy sensor, it can be used to determine a location of the occluded vessel. In such case, the driver 106 drives the transducer 420 to deliver diagnostic acoustic energy, and acoustic energy reflected from an object is sensed by the sensor and is analyzed to determine if the object is associated with an occluded vessel.

After a target vessel has been identified, the focused ultrasound device 400 can be used to recannalize the vessel. For such purpose, a blood thinning agent is first administered to the patient. Driver 106 then drives the transducer 420 to deliver treatment acoustic energy towards the target vessel. The treatment acoustic energy facilitates the blood thinning agent to dilate the vessel, thereby recannalizing the vessel, as similarly discussed previously.

In one embodiment, the transducer 420 is placed in contact with tissue, such as a vessel wall. While the transducer 420 applies treatment energy, the fluid source 130 pumps cooling fluid to the channel 412. The cooling fluid is delivered to the distal end 404 via the first fluid delivery lumen 410, and travels through the channel 412 to cool the surface 414, thereby preventing or at least reducing some of the heat that may be generated at the interface between the contacted tissue and the surface of the transducer 420. The cooling fluid carries heat away from the surface 414 and travels back to the fluid source 130 through the second fluid delivery lumen 411.

Alternatively, if bodily fluid, such as blood, is present, the fluid can be used as an acoustic coupling media. In such case, the transducer 420 can be placed away from a target tissue. Also, when bodily fluid is present, it can be used as cooling agent to carry heat away from the distal end 404.

Although the focused ultrasound device 400 has been described as having the channel 412 for delivering cooling fluid, in alternative embodiments, the cooling feature of the device 400 is optional. In such case, the device 400 does not include the channel 412 and the fluid source 130.

FIG. 5 shows another focused ultrasound device 500 which includes a handle 502 and a transducer 504 secured to the handle 502. The handle 502 includes a channel 508 beneath a surface 510 that is located adjacent to the transducer 504, a first fluid delivery lumen 506, and a second fluid deliver lumen 507. The first fluid delivery lumen 506 is configured to deliver fluid from the fluid source 130 to the channel 508, and the second fluid delivery lumen 507 is configured to return the delivered fluid back to the fluid source 130. In the illustrated embodiment, the fluid delivery lumens 506, 507 are located within a wall of the handle 502. Alternatively, the fluid delivery lumens 506, 507 can be implemented using tubes that are placed within the lumen of the handle 502.

In some embodiments, the focused ultrasound device 500 can further include one or more acoustic energy sensors 530 secured to the handle 502 for sensing acoustic energy that has been reflected from an object, such as tissue. The sensed acoustic energy can be processed and/or analyzed to determine whether a vessel has been identified, as similarly discussed previously.

In the illustrated embodiment, the handle 502 has a short profile (e.g., less than 12 inches in length), thereby allowing ease of positioning and/or aiming of the transducer 504 by a physician. During use, the focused ultrasound device 500 can be hand-held by a physician to aim towards a target tissue region. The transducer 504 can then deliver diagnostic and/or treatment acoustic energy to the target tissue region, as similarly discussed previously.

Although several embodiments have been described with reference to detecting vessels, such as cranial vessels, and recannalizing occluded cranial vessels, the scope of the invention is not so limited. In alternative embodiments, the above described devices (or similar devices) can be used to detect and/or treat vessels or tissue at other locations of a body. In addition, in alternative embodiments, instead of delivering recannalization acoustic energy, any of the transducers discussed previously can be configured to deliver acoustic energy at a level suitable for imaging or ablation of tissue.

Thus, although various embodiments have been shown and described, it would be apparent to those skilled in the art that many changes and modifications may be made thereunto without the departing from the scope of the invention, which is defined by the following claims.

What is claimed:

1. A method for performing ultrasound therapy in a target tissue region of a patient, the method comprising: placing a transducer on a tissue surface; delivering a blood thinning agent to a target tissue region underlying the tissue surface; delivering acoustic energy from the transducer to the target tissue region thereby causing dilatation of a vessel in the target tissue region; and cooling the tissue surface to remove heat generated from the delivered acoustic energy.

2. The method of claim 1, wherein the tissue surface comprises a skin surface.

3. The method of claim 1, wherein the tissue surface comprises dura tissue.

4. The method of claim 1, wherein the tissue surface a surface of an internal organ.

5. The method of claim 1, wherein the delivered acoustic energy is at a frequency between about two MHz and about ten MHz.

6. The method of claim 1, wherein the target tissue region comprises a brain vessel.

7. The method of claim 1, further comprising recording a neurological signal before or during the delivery of acoustic energy.
